(19) United States
(12) Patent Application Publication
(10) Pub. No.: US 2009/0101152 A1
(43) Pub. Date: Apr. 23, 2009

(54) High surface area anti-microbial coated endotracheal tube

(76) Inventors: Marc Burk, Murrieta, CA (US); Daniel Patrick Dwyer, Raleigh, NC (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/907,742

(22) Filed: Oct. 17, 2007

(51) Int. Cl. A61M 16/04

(52) U.S. Cl. 128/207.15

(57) ABSTRACT

The present invention provides a medical device such as an endotracheal tube or a tracheostomy tube having a distal inflation cuff and a high surface area structure disposed radially around the tube on a proximal side of the cuff. The high surface area structure includes an antimicrobial agent. The relatively higher surface area of the structure and radial extension thereof from the tube allows the antimicrobial agent to better extend into a secretion pool collected in the region around the intersection of the cuff and tube within a patient's trachea, so as to better prevent biofilms from forming and thereby prevent infections such as ventilator assisted pneumonia (VAP).

### FIELD OF THE INVENTION

The present invention relates generally to medical devices. More particularly, the present invention relates to artificial airways for insertion into the trachea which are coated with an antimicrobial agent.

### BACKGROUND OF THE INVENTION

This present invention addresses the problems incident with the insertion of medical devices such as endotracheal and tracheostomy tubes into a patient's airway. As is well known in the art, fluid secretions can pool around the distal cuff portion just proximal to the cuff around the tube when the device is dwelling inside a patient's trachea for an extended period of time. Infections, such as ventilator assisted pneumonia (VAP), can commonly result due to biofilm buildup around the device near or within the pool of secretions when using such a cuffed endotracheal or tracheostomy tube. Previous methods of preventing this buildup and the resultant infections have not been widely embraced in medical devices in the respiratory field due to their lack of effectiveness. There is a tremendous need to prevent or limit colonization of secretions that are believed to play a role in the development and prevalence of infections such as ventilator assisted pneumonia in patient's undergoing respiratory treatment or support.

Prior methods of preventing mucosal buildup around the cuff of an endotracheal tube include devices which incorporate a lumen in the endotracheal tube which can be attached to a suction device, such as marketed in Mallinckrodt's EVAC™ product line. Various devices covering endotracheal tubes with separate suction or evacuation lumens are disclosed in U.S. Pat. Nos. 4,305,392; 4,632,108; 4,637,389; 4,840,173; 5,143,062; 5,201,310; 5,311,864; 5,501,215; 5,520,175; 5,540,224; 5,819,723; 6,460,540; and 7,089,942. In the these suction lumen type devices, the intake port for suctioning secretions and mucous is generally located proximate to the point where the endotracheal tube and inflation cuff intersect, on the proximal side of the cuff. However, the secretions and mucous pooling at the junction of the tube and cuff can be large in volume and often remote from the suction port. Thus, these suction type devices are clinically limited in effectiveness to remove secretions as the suction port can be too remote from the main secretion buildup. Other suctioning systems place a device on the distal end of a ventilator circuit, allowing suctioning during ventilation.

Silver and antimicrobial coatings have been previously placed on various medical devices, including anesthesia, orthopedic and wound care products. As such, silver coatings and other antimicrobial coatings are well-established, safe and effective methods to limit biofilms and resultant infections on or near medical devices. Such antimicrobial coatings have also been known to be used with endotracheal or tracheostomy tubes, such as is disclosed in U.S. Pat. No. 5,725,510.

Accordingly, it is desirable to provide artificial airways such as an endotracheal or tracheostomy tube which minimizes the risk of infection while dwelling for extended periods of time in a patient. In particular it is specifically desirable to provide an apparatus that more effectively positions an antimicrobial coating proximate a secretion pool near a cuffed endotracheal tube to reduce the pathogen density within the secretion pool.

### SUMMARY OF THE INVENTION

The foregoing needs are met, to a great extent, by the present invention, wherein in one aspect an apparatus is provided that in some embodiments includes a high surface area structure attached to and/or near the proximal side of an endotracheal/tracheostomy tube cuff to increase the surface area of silver or other antimicrobial coatings to neutralize pathogens contained within the secretion buildup known to occur above the cuff area on artificial airways. This novel invention effectively positions an antimicrobial agent proximate or into the secretion pool and not just around the perimeter of an endotracheal tube or similar medical device, potentially reducing the pathogen density within the secretion pool leading to a reduction in infections such as VAP.

In accordance with one embodiment of the present invention, an endotracheal tube is provided including a tubular member having distal and proximal end portions. At least one inflatable cuff is disposed about the tubular member proximate the distal end portion. A high surface area structure is disposed about the tubular member and positioned proximal and adjacent to the cuff. The high surface area structure includes an antimicrobial agent.

In accordance with another aspect of the present invention, A medical device is provided, including an elongated tube having an inner lumen and distal and proximal end portions. At least one inflatable cuff is disposed about the elongated tube proximal to the distal end portion. A structure is disposed radially around the elongated tube on a proximal side of the cuff. The structure has a surface area and includes an antimicrobial agent.

In accordance with yet another aspect of the present invention, an endotracheal tube is provided having a distal end portion including at least one inflatable cuff. A means for increasing the surface area of the endotracheal tube is disposed at an intersection of the tube and cuff. An antimicrobial agent is included with at least a portion of the means for increasing the surface area of the endotracheal tube.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto. The invention can also include a means for evacuating fluid proximate to the cuff and proximal of the means for increasing the surface area of the endotracheal tube.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1A is a view illustrating an endotracheal or tracheostomy tube according to a first, preferred embodiment of the invention.

FIG. 1B is a cross-sectional view of the tube shown in FIG. 1A.

FIG. 2A is a view illustrating the endotracheal or tracheostomy tube according to the first embodiment of the invention with the cuff and high surface area structure in a non-inflated or contracted condition.

FIG. 2B is a cross-sectional view illustrating the endotracheal or tracheostomy tube according to the first embodiment of the invention with the cuff and high surface area structure in a non-inflated or contracted condition.

FIG. 3A is a view illustrating an endotracheal tube or tracheostomy tube according to a second embodiment of the invention.

FIG. 3B is a cross-sectional view of the device shown in FIG. 3A.

FIG. 4A is a view illustrating an endotracheal or tracheostomy tube according to a third embodiment of the invention.

FIG. 4B is a cross-sectional view of the endotracheal or tracheostomy tube shown in FIG. 4A.

FIG. 5A is a view illustrating an endotracheal or tracheostomy tube according to a fourth embodiment of the invention.

FIG. 5B is a cross-sectional view of the endotracheal or tracheostomy tube shown in FIG. 5A.

FIG. 6A is a view illustrating an endotracheal or tracheostomy tube according to a fifth or preferred embodiment of the invention.

FIG. 6B is a cross-sectional view of the endotracheal or tracheostomy tube shown in FIG. 6A.

### DETAILED DESCRIPTION

The invention will now be described with reference to the drawing figures, in which like reference numerals refer to like parts throughout. The present invention involves the use of an endotracheal/tracheostomy tube to minimize the concentration of pathogens within the secretion pool that typically collects on the proximal region around the cuff area. Various embodiments of the invention are arranged by placing structures such as flexible rings and/or discs in various sizes, shapes and forms near the proximal side of an inflatable cuff on an endotracheal or tracheostomy tube, thereby dramatically increasing the surface area of the medical device at the junction of the inflatable cuff and tube airway. Each of the structures disposed at the junction of the inflatable cuff and tube airway is characterized by a relatively high surface area or complex set of surfaces and geometries. The discs and other high surface area structures are made soft and flexible enough so as not to damage the tracheal wall and further can fill the volume or space near the proximal end of the endotracheal cuff.

In accordance with conventional practice, as used herein, the term “proximal” or “proximal end” shall refer to the specified end of a device or its component which is generally closer to the medical personnel handling or manipulating the device as it is intended to be used, and the term “distal” or “distal end” shall refer to the specified end of a device or its component which is opposite the proximal end.

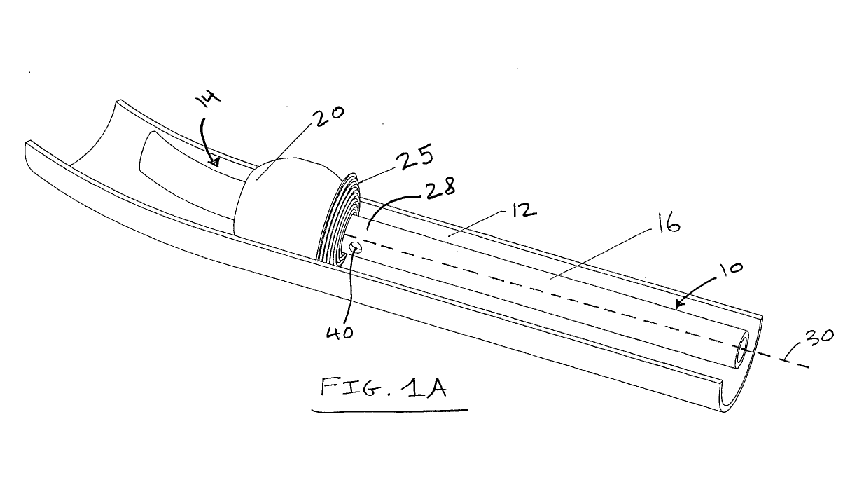

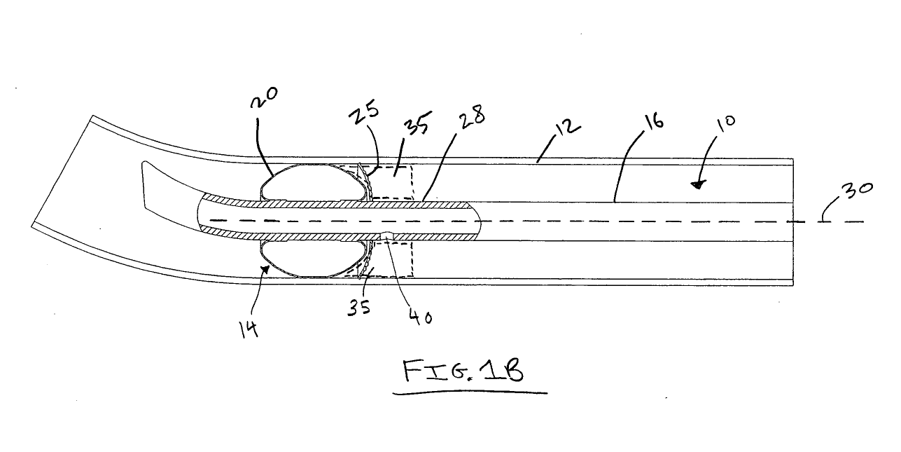

FIG. 1A is a schematic view illustrating an endotracheal or tracheostomy tube according to a first embodiment of the invention. A tube 10 is positioned inside a patient's trachea 12. The tube 10 includes a distal end portion 14 and a proximal end portion (not shown) which generally extends out of the patient's oral airway opening when the tube 10 is inserted as shown. The tube 10 includes an elongated tubular member 16 which defines one or more lumens therein for providing an artificial airway as is well-known in the art. A distal cuff 20 is disposed at a point on or proximate the distal end portion 14 of the tube 10. The cuff 20 is an inflatable member which is used to position and secure the tube 10 when inserted into a patient's trachea, as is well known in the art.

On the proximal end or side of the cuff sub-assembly is a high surface area structure 25, having the general configuration or geometry shown in FIG. 1A. As used herein, the term “high surface area structure” shall mean any structure having a relatively high surface area, including, but not limited to, a relatively high surface area to volume ratio, above that of the spherical surface of inflatable cuffs or cylindrical surface of airway tubes commonly used in the art for endotracheal/tracheostomy tubes. The “high surface area structure” can also mean any series of complex curved surfaces or structures that extend radially out from the outer surface 28 and away from a central longitudinal axis 30 of the tube device 10. In the preferred embodiment shown in FIG. 1 A, the high surface area structure 25 is a pleated thin sheet of material, such as a plastic, which can expand and contract with the inflation and deflation, respectively, of the cuff 20.

A portion of the outer surface of the distal end portion 14 of the tube 10, the tubular member 16, the cuff 20, and especially the high surface area structure 25, includes an anti-microbial agent. As used herein, the term “anti-microbial agent” shall mean any material, substance, or compound, such as any number of silver containing materials, or other oligodynamic materials or compounds, which provide anti-infective properties as is well known in the art. As used herein, the “inclusion” of the anti-microbial agent with the present inventive apparatus may involve, without limitation, coating, treating, compounding, or materially incorporating the anti-microbial agent onto or into the structure and/or surface of the distal end portion 14 of the tube 10, the tubular member 16, the cuff 20, and preferably the high surface area structure 25. The anti-microbial agent used with the present invention is especially effective when coated on the outer surface of the high surface area structure 25, as further illustrated in FIG. 1B.

FIG. 1B shows a schematic cross-sectional view of the tube 10 shown in FIG. 1A. The pleated high surface area structure 25 forms an annular disc structure which extends radially away from the outer surface 28 of the tubular member 16 into the space between the tubular member 16 and the inside walls of the patient's trachea 12. The high surface area structure 25 is disposed on the proximal end or side of the cuff 20. Regions 35 indicate one of the potential spaces in which secretions can collect when the tube 10 is inserted for extended periods, and which can lead to formation of biofilms and resulting infections such as VAP. As shown in FIG. 1B, the high surface area structure 25 extends into the region 35 and provides a series of additional complex surfaces from which anti-microbial agents can contact the secretions and thereby limit the proliferation of biofilms and infective organisms. The high surface area structure 25 accomplishes this in two ways: (i) by extending into the area 35 or space between the tube 16 and trachea wall 12 where secretions normally accumulate, and (ii) by providing additional surface area above and beyond what is normally provided by the outer surfaces of the tube 16 and cuff 20.

As shown in FIGS. 1A and 1B, the present inventive apparatus 10 can also include a suction or intake port 40 for suctioning secretions via a separate suction or evacuation lumen (not shown) incorporated into the tube device, as is well-known in the art. The suction or intake port 40 can be located on the proximal side of the tube from the high surface area structure 25 for maximum effectiveness.

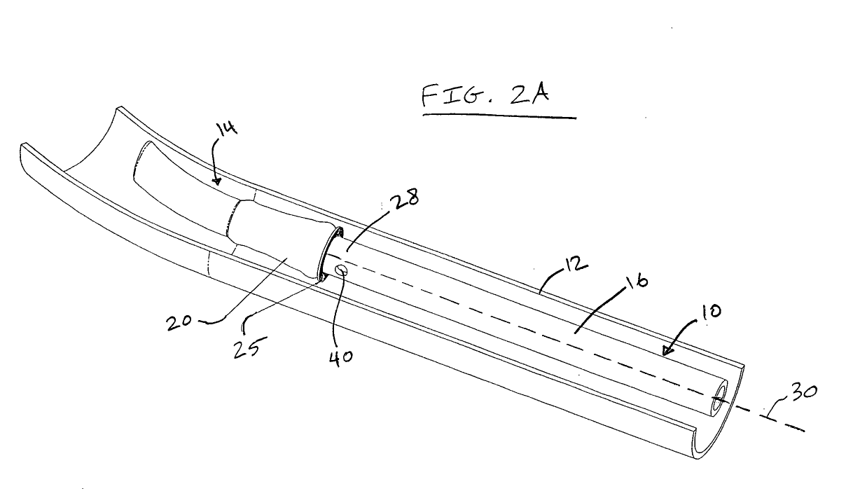

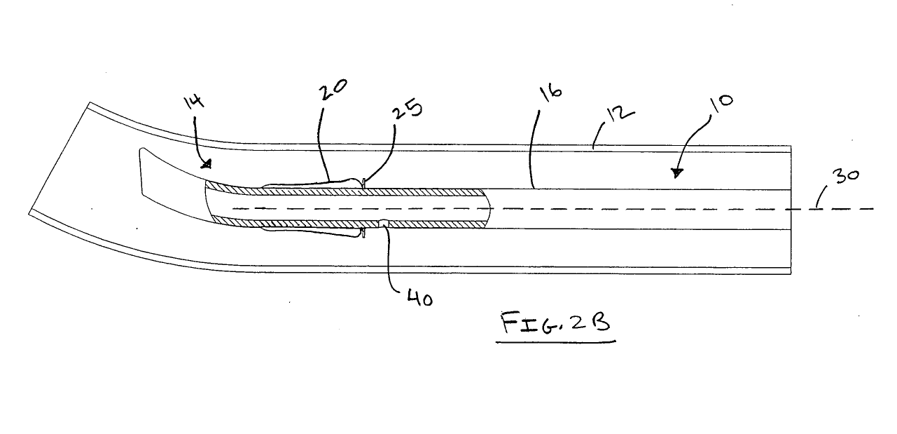

FIG. 2A is a schematic view illustrating the endotracheal or tracheostomy tube 10 with the cuff 20 and high surface area structure 25 in a non-inflated condition. The high surface area structure 25 included in this embodiment of the present invention is a pleated structure which can expand when the cuff 20 is inflated and which can contract when the cuff 20 is deflated, as shown in FIG. 2A. FIG. 2B is a schematic cross-sectional view illustrating the endotracheal or tracheostomy tube 10 with the cuff 20 and high surface area structure 25 in a non-inflated condition as in FIG. 2A. The expansion and contraction of the cuff and high surface area structure 25 allows for safe insertion of the tube 10 into a patient's trachea without damaging tissue.

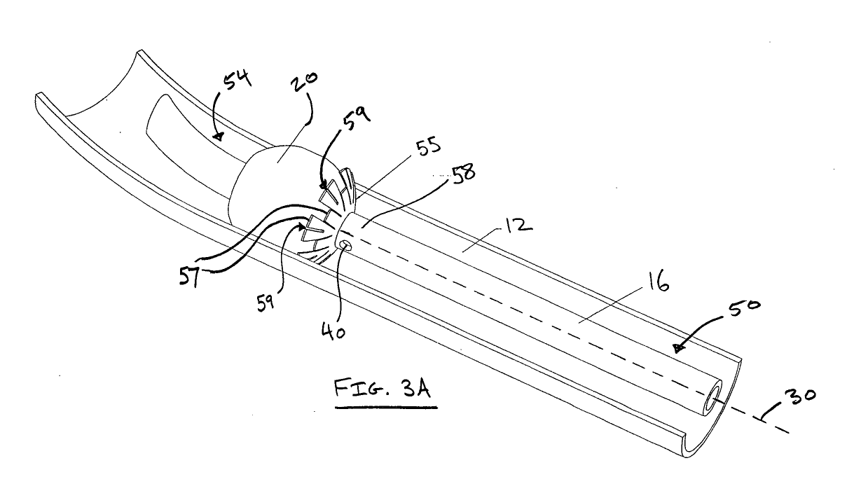

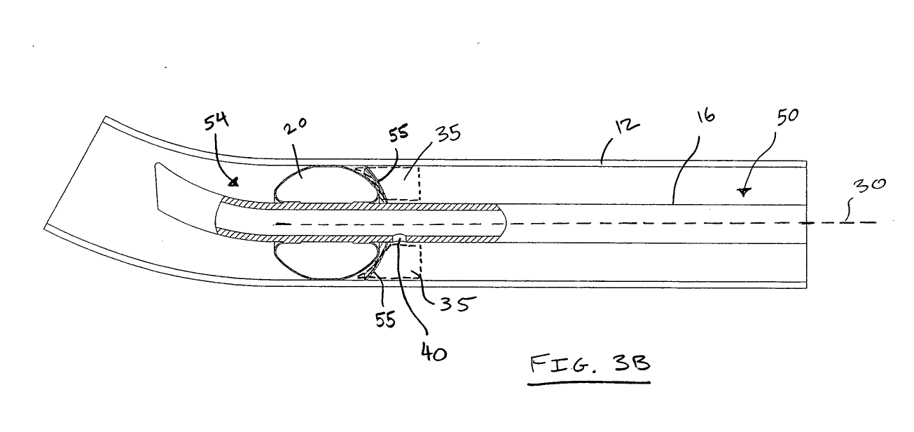

FIG. 3A is a schematic view illustrating an endotracheal tube or tracheostomy tube 50 according to a second embodiment of the invention. Tube 50 includes the same tubular member 16 and cuff 20 as tube 10, but the distal end portion 54 includes a high surface area structure 55 which includes a soft disc shaped structure forming an annulus around the outer surface 58 of the tubular member 16 proximate the junction of the cuff 20 with tube 16. As shown in FIG. 3A, the disc of the high surface area structure 55 includes a plurality of slits 59 cut into the disc that form radially extending strips 57 to allow for more flexibility in the structure such that the tube 50 can be safely inserted into the trachea. This also allows the high surface area structure 55 to bend or collapse as the cuff is deflated or when the tube 50 is inserted. The high surface area structure 55 also includes an anti-microbial agent to allow for the tube device 50 to more effectively prevent the formation of biofilms and infections in the space 35 proximate the high surface area structure 55 on the proximal side of the cuff 20. FIG. 3B is a schematic cross-sectional view of the device shown in FIG. 3A. As shown in both FIGS. 1B and 3B, the high surface area structures of annular pleated disc 25 or slitted disc 55 have a radial diameter extending from axis 30 which at least equals, or can exceed, the radial diameter of the inflatable cuff 20 in its fully inflated condition.

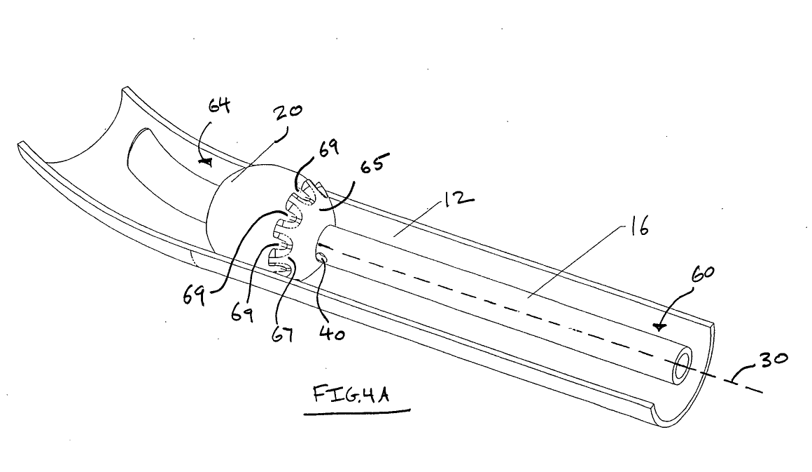

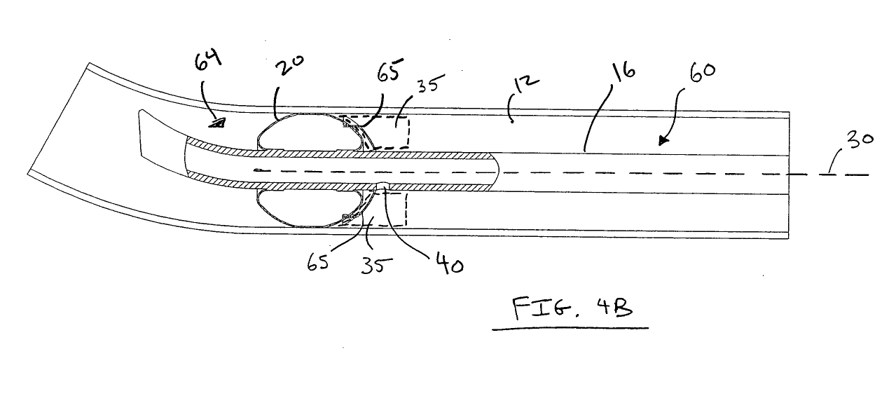

FIG. 4A is a schematic view illustrating an endotracheal or tracheostomy tube 60 according to a third embodiment of the invention. Tube 60 includes the same tubular member 16 and cuff 20 as tubes 10 or 50, but the distal end portion 64 includes a high surface area structure 65 which includes an anti-microbial agent and is a dome-shaped element defining a plurality of recesses 69 within the outer surface 67 of the dome-shaped element. The dome-shaped element 65 overlies a proximal side of the inflatable cuff 20. FIG. 4B is a cross-sectional view of the endotracheal or tracheostomy tube shown in FIG. 4A. As shown in FIG. 4B, the dome shaped structure 65 extends radially away from central axis 30 and into region 35 to provide a greater contact area for secretions. The plurality of recesses 69 as well as the radial girth of the dome shaped element 65 both contribute to this increased surface area.

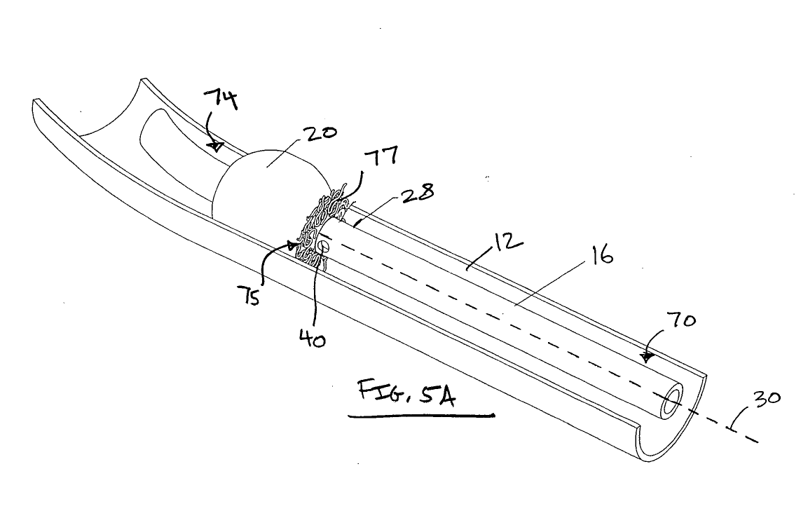

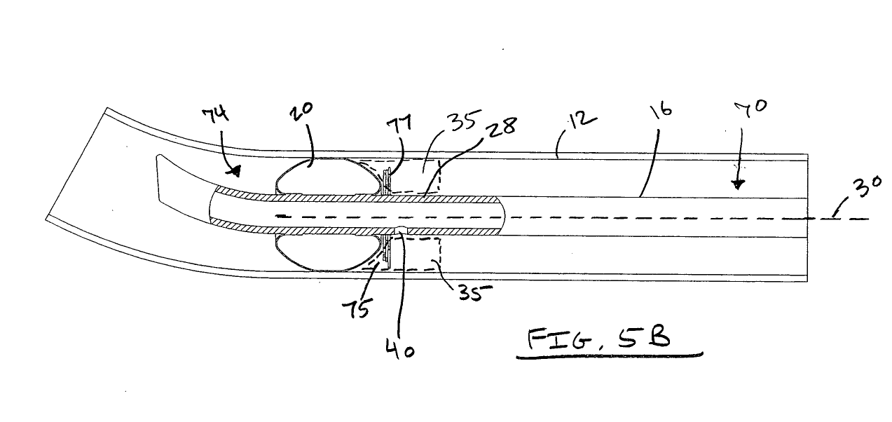

FIG. 5A is a schematic view illustrating an endotracheal or tracheostomy tube 70 according to a fourth embodiment of the invention. Tube 70 includes the same tubular member 16 and cuff 20 as tubes 10, 50, or 60, but the distal end portion 74 includes a high surface area structure 75, which includes an anti-microbial agent, and which includes a plurality of curved or curly hair-like or fiber-shaped elements extending radially from an outer surface of the tubular member, as shown in FIG. 5A. Each fiber-shaped element can have a plurality of bends to present a complex geometry and set of complex curves which creates a relatively high surface area from which the anti-microbial agent applied thereon can more efficiently contact pathogens and thereby more effectively limit biofilm formation and infections. FIG. 5B is a cross-sectional view of the endotracheal or tracheostomy tube 70 shown in FIG. 5A, which shows the fiber structures 77 on high surface area structure 75 extending radially into the secretion pooling region 35, similar to the way high surface area structures 25, 55, and 65 extend into region 35.

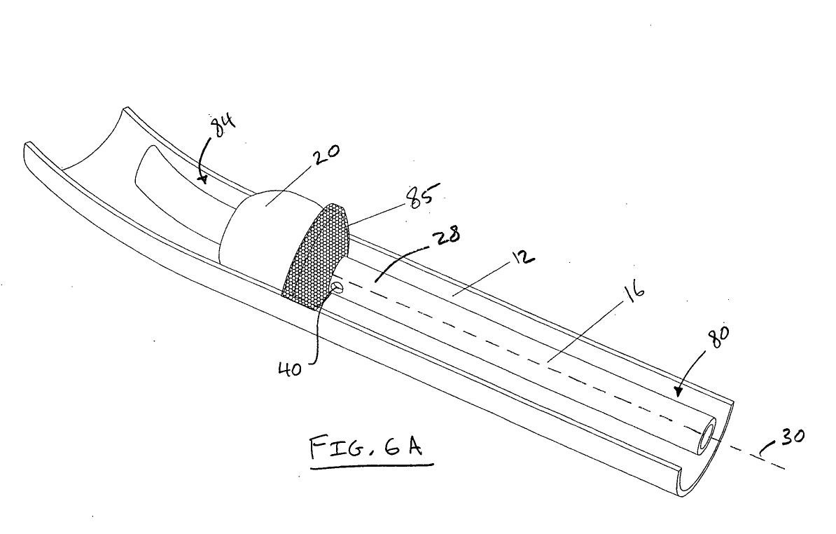

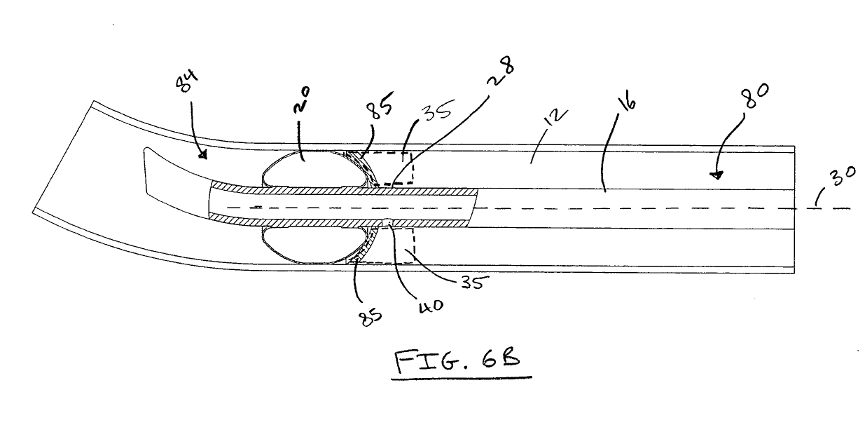

FIG. 6A is a schematic view illustrating an endotracheal or tracheostomy tube 80 according to a fifth or preferred embodiment of the invention. Tube 80 includes the same tubular member 16 and cuff 20 as tubes 10, 50, 60, or 70, but the distal end portion 84 includes a high surface area structure 85 which includes an anti-microbial agent, and which structure 85 includes or forms a spongy material. The spongy material of high surface area structure 85 inherently includes a great plurality of inner complex curved surfaces that are characteristic of any spongy material. This embodiment of the present invention provides both a greatly increased surface area structure in spongy structure 85 but also enables a greater volume or amount of anti-microbial agent to be applied or stored on the tube device 80. FIG. 6B is a schematic cross-sectional view of the endotracheal or tracheostomy tube shown in FIG. 6A. The spongy high surface area structure 85 can also extend radially into region 35 and can have a radial diameter at least as great as that of the inflatable cuff 20. The spongy nature of high surface area structure 85 also has the added benefit of being able to easily and reversibly contract and expand as the tube 80 is inserted into the trachea for added patient safety.

The parts utilized for the present invention can include conventional soft, thin plastic resins that are pleated or shaped to create a high surface area structure. The parts may also be made of fibrous, tentacle or hair-like structures. The high surface area plastic, film, media or other structures, such as structures 25, 55, 65, 75 or 85 are generally adhered near to or on the proximal side of the endotracheal tube cuff 20. However, the high surface area structures could also be integrally molded or extruded to the cuff 20 and/or tube 10, 50, 60, 70, or 80, or affixed through solvent bonding, liquid adhesion or other common bonding techniques. Flexible materials may also be stretched over the tube and held in place through interference. Materials can be pleated or formed to create the greatest surface area possible. Materials of construction are soft or fibrous and also can incorporate a thin wall to prevent tracheal damage.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. An endotracheal tube, comprising:
a tubular member having distal and proximal end portions;
at least one inflatable cuff disposed about the tubular member proximate the distal end portion; and
a high surface area structure disposed about the tubular member and positioned proximal and adjacent to the cuff, the high surface area structure including an antimicrobial agent.

2. The endotracheal tube of claim 1, further comprising:
an evacuation lumen disposed within the tubular member having an intake port defined by walls of the tubular member and disposed proximal of and adjacent to the high surface area structure.

3. The endotracheal tube of claim 1,
wherein the high surface area structure comprises a pleated annular disc.

4. The endotracheal tube of claim 3,
wherein the pleated annular disc is expandable with the inflatable cuff.

5. The endotracheal tube of claim 3,
wherein the pleated annular disc has a radial diameter equal to a maximum radial diameter of the inflatable cuff.

6. The endotracheal tube of claim 1,
wherein the high surface area structure comprises a flexible annular disc.

7. The endotracheal tube of claim 6,
wherein the flexible annular disc comprises a plurality of radially extending strips separated by slits defined by the disc.

8. The endotracheal tube of claim 1,
wherein the high surface area structure comprises a dome-shaped element defining a plurality of recesses within an outer surface of the dome-shaped element, the dome-shaped element overlying a proximal side of the inflatable cuff.

9. The endotracheal tube of claim 1,
wherein the high surface area structure comprises a plurality of fiber-shaped elements extending radially from an outer surface of the tubular member, each fiber-shaped element having a plurality of bends.

10. The endotracheal tube of claim 1,
wherein the high surface area structure comprises a sponge-like material.

11. A medical device, comprising:
an elongated tube having an inner lumen and distal and proximal end portions;
at least one inflatable cuff disposed about the elongated tube proximal to the distal end portion; and
a structure disposed radially around the elongated tube on a proximal side of the cuff, the structure having a surface area and including an antimicrobial agent.

12. The medical device of claim 11, further comprising:
an evacuation lumen disposed within the elongated tube having an intake port defined by walls of the tubular member and disposed proximal of and adjacent to the structure.

13. The medical device of claim 11,
wherein the structure comprises a pleated annular disc.

14. The medical device of claim 13,
wherein the pleated annular disc is inflatable with the inflatable cuff.

15. The medical device of claim 13,
wherein the pleated annular disc has a radial diameter equal to a maximum radial diameter of the inflatable cuff.

16. The medical device of claim 11,
wherein the structure comprises a flexible annular disc.

17. The medical device of claim 16,
wherein the flexible annular disc comprises a plurality of radially extending strips separated by slits defined by the disc.

18. The medical device of claim 11,
wherein the structure comprises a dome-shaped element defining a plurality of recesses within an outer surface of the dome-shaped element, the dome-shaped element overlying a proximal side of the inflatable cuff.

19. The endotracheal tube of claim 11,
wherein the structure comprises a plurality of fiber-shaped elements extending radially from an outer surface of the tubular member, each fiber-shaped element having a plurality of bends.

20. The endotracheal tube of claim 1,
wherein the structure comprises a sponge-like material.

21. An endotracheal tube, comprising:
a tube having a distal end portion including at least one inflatable cuff;
a means for increasing the surface area of the endotracheal tube disposed at an intersection of the tube and cuff; and
an antimicrobial agent included with at least a portion of the means for increasing the surface area of the endotracheal tube.

22. The endotracheal tube of claim 21, further comprising:
a means for evacuating fluid proximate to the cuff and proximal of the means for increasing the surface area of the endotracheal tube.
